(19) United States
(12) Patent Application Publication
(10) Pub. No.: US 2020/0375915 A1
(43) Pub. Date: Dec. 3, 2020

(54) MATRIX ADHESIVE PATCH AND PROCESS FOR THE PREPARATION THEREOF

(71) Applicants: Srinivas Reddy MALE, Hyderabad (IN); Pravin Bhikan Rao PATIL, Hyderabad (IN); Ganesh Haridas DOHE, Hyderabad (IN); Shantaram Laxman PAWAR, Hyderabad (IN); Azista Industries Pvt Ltd, Hyderabad (IN)

(72) Inventors: Srinivas Reddy Male, Hyderabad (IN); Pravin Bhikan Rao Patil, Hyderabad (IN); Ganesh Haridas Dohe, Hyderabad (IN); Shantaram Laxman Pawar, Hyderabad (IN)

(21) Appl. No.: 16/607,313

(22) PCT Filed: Apr. 25, 2018
(86) PCT No.: PCT/IB2018/052845

§ 371 Date: Oct. 22, 2019

(30) Foreign Application Priority Data
Apr. 25, 2017 (IN) 201741014663

(51) Int. Cl. A61K 9/70; A61K 31/045; A61K 31/125; A61K 31/618

(52) U.S. Cl. CPC A61K 9/7076; A61K 31/618; A61K 31/125; A61K 31/045

(57) ABSTRACT

The present invention relates to matrix adhesive patch and its preparation. The present invention specifically relates to an analgesic matrix adhesive patch and its preparation. The present invention more specifically relates to a matrix patch adhesive preparation containing Camphor, Menthol and Methyl salicylate and process for the preparation thereof which delivers predetermined amount of drug continuously through skin and provide the temporary relief of minor aches & pains of muscles & joints associated with; Arthritis, Strains, Bruises & Sprains.

### FIELD OF THE INVENTION

The present invention relates to matrix adhesive patch and its preparation.

The present invention specifically relates to an analgesic matrix adhesive patch and its preparation.

The present invention more specifically relates to a matrix patch adhesive preparation containing Camphor, Menthol and Methyl salicylate and process for the preparation thereof which delivers predetermined amount of drug continuously through skin and provide the temporary relief of minor aches & pains of muscles & joints associated with Arthritis, Strains, Bruises & Sprains.

### BACKGROUND OF THE INVENTION

The primary use of a topical analgesic is to relieve the pain such as associated with arthritis as well as muscle aches and pains caused by sports injuries or physical work. One benefit of topical pain relievers is that they can be apply directly to the site of the pain, so there is minimal systemic distribution of the pain reliever throughout the body. This localized application and associated action minimizes the potential for systemic side effects. In addition, the pain relieving action of topical analgesics is faster than most oral forms because it is applied directly onto the painful area whereas oral analgesics need to be dissolved, absorbed in the gastrointestinal tract, survive first-pass metabolism in the liver and then be transported throughout the body.

However, the matrix adhesive preparation containing methyl salicylate supress pain by blocking the enzymes involved in the production of prostaglandins, which signal inflammation and cause pain, Menthol has analgesic properties that are mediated through a selective activation of κ-opioid receptors and Camphor is readily absorbed through the skin and produces a feeling of cooling similar to that of menthol, and can act as a local anaesthetic substance.

The advantage of Topical patch over the topical gel is that, it delivers the sustained release of drug into systemic circulation in one application rather than frequent application of topical gel and produces the sustained therapeutic effect.

U.S. Pat. No. 8,809,615 B2 discloses that the manufacturing process of adhesive preparation by hot melt technique and the adhesive base was coated on interlock woven fabric and the support should have a weight of 80 to 150 g/m².

U.S. Pat. No. 4,147,831 A discloses a pressure-sensitive adhesive composition having improved adhesion characteristics in water comprising an elastomeric mixture consisting of natural rubber and polyisobutylene, a liquid plasticizer component and a solid tackifier component.

U.S. Pat. No. 5,508,038 A discloses an in-line adhesive, useful for transdermal delivery devices comprising a mixture of high and low molecular weighted polyisobutylene which is substantially free of plasticizers and tackifiers.

U.S. Pat. No. 5,866,157 A discloses a matrix type patch formulation which comprises an adhesive layer containing a physiological active substance, an organic acid, a hydrophobic high molecular material, a tackifying resin, a plasticizer and an absorption enhancer.

U.S. Pat. No. 8,734,838 B2 discloses a pressure-sensitive adhesive composition which comprises three ingredients, i.e., polyisoprene, a styrene/isoprene/styrene copolymer, and solid polyisobutylene and further contains a non-solid isobutylene polymer and a tackifier. This document also discloses that this preparation comprises one or more analgesic anti-inflammatory agents selected from the group consisting of methyl salicylate, L-menthol and dl-camphor.

International Journal of Drug Delivery 1, 2009, 41-51 discloses a matrix-type transdermal therapeutic system containing drug Aceclofenac with different ratios of hydrophilic and hydrophobic polymeric systems by the solvent evaporation technique by using plasticizer.

All the prior art references related to the adhesive transdermal patches and process for the preparation thereof. However, the adhesive preparation of present invention has been prepared, to ease of manufacturing of adhesive preparation containing volatile drugs by improving the manufacturing process by using solvent evaporation technique and reduces the loss of drugs during manufacturing process like hot melt technique. The adhesive preparation of present invention has been prepared by using low boiling point straight chain alkane solvents to reduce the loss of drugs during manufacturing process. The purpose of use of low boiling solvent to minimize the drying temperature & time, this leads to reduce the drug loss from the adhesive matrix during drying process.

### OBJECTIVE OF INVENTION

The objective of the present invention is to provide a matrix adhesive patch composition and process for preparation thereof.

Another objective of the present invention is to provide an improved manufacturing process for preparation of matrix adhesive patch by using solvent evaporation technique, which reduces the loss of drugs during manufacturing process.

Another objective of the present invention is to provide use of low boiling solvent to minimize the drying temperature & time, this leads to reduce the drug loss from the adhesive matrix during drying process.

### SUMMARY OF INVENTION

One embodiment of the present invention is to provide a matrix adhesive patch composition.

Another embodiment of the present invention is to provide an improved process for preparing a matrix adhesive patch.

Another embodiment of the present invention is to provide a matrix adhesive patch composition comprising Camphor, Menthol, Methyl salicylate and one or more other excipients.

Another embodiment of the present invention is to provide an improved manufacturing process for preparation of matrix adhesive patch preparation which involves solvent evaporation technique to reduce the loss of drugs during manufacturing process.

Another embodiment of the present invention is to provide an improved manufacturing process for preparation of matrix adhesive patch, which involves use of low boiling solvent to minimize the drying temperature & time, this leads to reduce the drug loss from the adhesive matrix during drying process.

Another embodiment of the present invention provides a matrix adhesive patch composition comprising Camphor, Menthol, Methyl salicylate and one or more other excipients selected from adhesive material, tackifying resin, adsorbent material and antioxidants.

Another embodiment of the present invention provides a matrix type patch composition comprising;

1% to 50% of one or more active ingredients,
50% to 80% (w/w) of adhesive material,
5% to 10% (w/w) of the tackifying resin,
3 to 5% (w/w) of adsorbent material,
& 0.5% to 2.0% of the antioxidants, and
1 to 90% (w/w) of other excipients.

Another embodiment of the present invention provides a matrix type patch composition comprising;

1% to 10% of camphor,
1% to 20% of menthol,
1% to 20% of methyl salicylate,
50% to 80% (w/w) of the combination of hydrophobic Low & high molecular weight adhesive material,
5% to 10% (w/w) of the tackifying resin,
3 to 5% (w/w) of adsorbent material,
0.5% to 2.0% of the antioxidants, and
1 to 90% (w/w) of other excipients.

Another embodiment of the present invention provides a matrix type patch composition comprising;

1% to 10% of camphor,
1% to 20% of menthol,
1% to 20% of methyl salicylate,
50% to 80% (w/w) of Polyisobutylenes,
1% to 10% of mineral oil,
5% to 20% of Alicyclic saturated hydrocarbon resin,

### 1% to 10% of Aerosil, and

1 to 90% (w/w) of other excipients.

Yet another embodiment of the present invention provides a process for preparing a matrix type patch formulation, the process comprising steps of:
a. dissolving the combination of hydrophobic high molecular weight & low molecular adhesive material in one or more organic solvents,
b. adding one or more active ingredients, tackifying resins, antioxidants by stirring the mixture to obtain a uniform blend of adhesive base,
c. coating the adhesive base uniformly on to the silicone coated release liner and drying at low temperature for evaporation of organic solvents,
d. laminating the dried adhesive matrix by 100% polyester Nonwoven or woven fabric, and
e. cutting the resulting product into a desired size to producing an adhesive patch.

### DETAILED DESCRIPTION OF THE INVENTION

As is evident from the description of Prior-art, the manufacturing process of formulation was by hot melt technique. In this, all the excipients were first heat mixed at their respective proportions at 150° C. to 200° C. Then at same process temperature, drug was added thereto and mixture was stirred to obtain a uniform melt. Pertaining to this process, adding volatile drugs at 150° C. to 200° C., causes loss of high amount of drugs. Therefore, even if a usual adhesive preparation is supplemented with high concentration of drugs to compensate the loss during manufacturing, it is difficult to maintain the drugs at high concentration in adhesive preparation, due to volatilization and even the hot melt process seems very tedious or complicated to manufacture the formulation. However, such an approach is possible but to compensate the high amount of loss of volatile drugs, extra amount of drugs needs to be add (Overages). This leads to increase in the cost of products. However, such approach will work better for those drugs, which are thermostable, having higher melting point than the thermoplastic polymer used in formulation and the Active ingredient that does not hinder hardening of molten formulation mass during manufacturing process.

Further, it is evident from the Prior-art that, the melted adhesive base coated on the interlock woven fabric backing membrane and the weight for this has been specified between 80 to 150 g/m² range. If the weight of woven fabric does not falls within this range, an adhesive base will seep through the stitches of knitted fabric. Therefore, selecting such type of backing membrane to coat the adhesive base on it produced the limitation of use of different type of backing membranes. However, such an approach is possible, but there is uncertainty to get the finished product with desired properties.

The adhesive preparation of present invention has been prepared in consideration of the problems described above. Specifically, an object of the present invention is to provide an adhesive preparation, which contains the therapeutically effective amount of drugs in an adhesive and capable of exhibiting percutaneous absorption and produce sufficient anti-inflammatory analgesic effects.

The adhesive preparation of present invention has been prepared by solvent evaporation technique using straight chain alkane solvent to minimize the loss of high amount of drugs during the process of manufacturing. The advantage of Solvent evaporation technique over Hot melt technique, it is simple and easy to manufacture and the loss of drugs during manufacturing is minimize by drying the adhesive layer at low temperature in minimum time.

The adhesive preparation of present invention has been prepared by coating the adhesive preparation on silicone coated release liner and matrix layer is laminated by Stretchable nonwoven or woven fabric backing material. The limitation of use of specified backing membrane as per Prior-art has been ruled out. In addition, the chance of seep of adhesive base through woven fabric backing membrane, when coated on to has been rectified as Prior-art by coating the adhesive base on PET, silicone coated release liner.

The adhesive preparation of present invention is an adhesive preparation comprising a stretchable support and the support used in present invention is an stretchable nonwoven or woven fabric backing is knitted fabric comprising 100% Polyethylene terephthalate less interacting with drug is more preferable. This knitted fabric is sufficiently stretchable. Therefore, the adhesive preparation of present invention can firmly follow the skin without lift off or dropping off when applied locally, for example, applied to the knee or elbow.

The adhesive material referred above preferably include hydrophilic polymers include, light anhydrous silicic acid, cellulose derivatives [carboxymethyl cellulose (CMC), carboxymethyl cellulose sodium (CMCNa), methyl cellulose (MC), hydroxypropylmethyl cellulose (HPMC), hydroxypropyl cellulose (HPC), hydroxyethyl cellulose (HEC)], starch derivatives (pullulan), polyvinyl alcohol (PVA), polyvinylpyrrolidone (PVP), vinyl acetate (VA), carboxyvinyl polymer (CVP), ethylvinyl acetate (EVA), Eudragit, gelatin, polyacrylic acid, sodium polyacrylate, polyisobutylene, combination of hydrophobic Low & high molecular weight polyisobutylene, polyisobutylene-maleic anhydride copolymer, alginic acid, sodium alginate, carrageenan, Arabian gum, tragacanth gum, mineral oil, karaya gum and polyvinyl methacrylate. In particular, polyisobutylene and combination of hydrophobic Low & high molecular weight polyisobutylene.

The tackifying resin referred above preferably includes an alicyclic saturated hydrocarbon resin, a poly-terpene resin, petroleum resin, rosin, rosin ester and fat soluble phenol resin or combinations thereof. The alicyclic saturated hydrocarbon resin is particularly preferable.

The adsorbent material referred above preferably includes colloidal silica, Aerosil, talc Al₂O₃, TiO₂, MgO, synthetic and amorphous silicas, Syloid, and synthetic and amorphous silicates and combinations thereof. Colloidal silicon dioxide is particularly preferable.

The antioxidant referred above preferably includes butylhydroxytoluene (BHT), butylhydroxyanisole (BHA), tertiary-butylhydroquinone (TBHQ), Tocopheryl acetate, alpha tocopherol, ascorbic-acid, ascorbyl palmitate, propyl gallate, fumaric acid, malic acid, sodium ascorbate, sodium metabisulfite, and combinations thereof. The butyl hydroxytoulene (BHT) & Tocopheryl acetate is particularly preferable.

The solvents referred above preferably includes Butane, Pentane, Hexane, Heptane, Octane and combinations thereof.

The other excipients referred above preferably includes plasticizers, solvents, stabilizer, surfactant, reducing agent, base, filler, coloring matter, softener, excipient, antiseptics, preservative, solubilizing agent, solubilizer, solvent, super plasticizer, antistatic agent, extender, moisturizing agent, and the like.

For the matrix type adhesive preparation of the present invention, it is preferred that the adhesive layer should contain a thermoplastic elastomer. The thermoplastic elastomer is easy to handle and causes a relatively low irritation to the skin. Therefore, it is preferably used. It is preferred that, the thermoplastic elastomer should be kind of Polyisobutylene (PIB) and Styrene isoprene styrene (SIS) block copolymers. The styrene isoprene styrene (SIS) block copolymer is soluble in high boiling point solvents such as toluene, benzene, methyl ethyl ketone. Using these solvents, preparation of adhesive matrix with Styrene isoprene styrene (SIS) block copolymer and volatile drugs such as Camphor, menthol & methyl salicylate will be possible. But, to get the residual solvent within adhesive matrix in limit as per ICH Q3C, adhesive matrix has to dry at higher temperature and at higher temperature the loss of drug is more due to volatilization. Hence, to reduce the loss of drug, adhesive matrix should be dry at low temperature and this is possible only by using the low boiling solvents and the SIS block polymer is not soluble in low boiling point solvents such as Pentane, Hexane, Heptane & Octane. The rationale of use of these straight chain alkane solvents is that, all are low boiling point solvents & the Polyisobutylene (PIB) is completely soluble in all these solvents. The preparation of adhesive matrix by solvent evaporation technique using these solvents, the loss of drugs during manufacturing process is being minimized. Hence, it is particularly preferred that, the adhesive preparation comprising Polyisobutylene and low molecular weight straight chain alkane solvent.

It is preferred that, the adhesive preparation comprising the Polyisobutylene, but the hardness, adhesive & cohesive property of adhesive matrix is not achieved with alone low molecular weight Polyisobutylene or high molecular weight Polyisobutylene. These physical & mechanical properties of matrix is achieved only with the combination of low molecular & high molecular weight PIB with the ratio of 1:1 & to get the desired tackiness & flexibility to matrix, the alicyclic saturated hydrocarbon resin is being added.

The physical property of adhesive matrix i.e. Tack adhesion of Test product and Reference product was found similar, the value was between 3.3 newton to 3.5 newton tested by probe tack method using Universal Testing machine. With application of Test product to human skin for 8 hrs, after removal of patch from skin, no residue was remained on the skin.

It is preferred that, the content of Polyisobutylene should contain 50% to 80% by mass with respect to total mass of adhesive layer. If the content falls within this range, the cohesive property of adhesive layer can be maintained. Accordingly, favourable application properties can be obtained.

Since appropriate tackiness and flexibility can be imparted to the adhesive preparation, it is preferred that the adhesive layer should contain an alicyclic saturated hydrocarbon resin as a tackifier. It is preferred that, the content of tackifier should be 5% to 10% by mass with respect to the total mass of adhesive layer. If the content falls within this range, the adhesion of adhesive layer can be maintained. Furthermore, the resulting adhesive preparation can be prevented from lift off when applied to the skin or from causing pain when peeled off.

For the matrix type adhesive preparation of present invention, it is preferred that the organic solvent used is a straight chain alkane comprising Pentane, Hexane, Heptane & Octane alone or in combination. It is preferred that, the organic solvent should meets the requirement of residual solvent limit as per ICH Q3C in final adhesive matrix patch.

For the matrix type adhesive preparation of present invention, it is preferred that the adhesive layer should contain antioxidants. Tocopheryl acetate & butylated hydroxyl toluene (BHT) are desirable as such as antioxidant. It is preferred that, the content of antioxidant should be between 0.5% to 2.5% by mass with respect to total mass of adhesive layer.

The following examples describes the nature of the invention and are given only for the purpose of illustrating the present invention in more detail and are not limitative and relate to solutions which have been particularly effective on a bench scale:

### Example 1

| S. No | Name of the ingredient | Percentage w/w |
|---|---|---|
| 1. | Camphor | 1.2% |
| 2. | Menthol | 5.7% |
| 3. | Methyl salicylate | 6.3% |
| 4. | Polyisobutylene | 66.3% |
| 5. | Alicyclic saturated hydrocarbon resin | 10% |
| 6. | Mineral Oil | 3% |
| 7. | Aerosil | 5.0% |
| 8. | Butylated Hydroxy toluene | 0.5% |
| 9. | Tocopheryl acetate | 2.0% |
| 10. | Pentane | q.s |

| | |
|---|---|
| Total | 100% |

### Example 2

| S. No | Name of the ingredient | Percentage w/w |
|---|---|---|
| 1. | Camphor | 1.2% |
| 2. | Menthol | 5.7% |
| 3. | Methyl salicylate | 6.3% |
| 4. | Polyisobutylene | 66.3% |
| 5. | Alicyclic saturated hydrocarbon resin | 10% |
| 6. | Mineral Oil | 3% |
| 7. | Aerosil | 5.0% |
| 8. | Butylated Hydroxy toluene | 0.5% |
| 9. | Tocopheryl acetate | 2.0% |
| 10. | Hexane | q.s |

| | |
|---|---|
| Total | 100% |

### Example 3

| S. No | Name of the ingredient | Percentage w/w |
|---|---|---|
| 1. | Camphor | 1.2% |
| 2. | Menthol | 5.7% |
| 3. | Methyl salicylate | 6.3% |
| 4. | Polyisobutylene | 66.3% |
| 5. | Alicyclic saturated hydrocarbon resin | 10% |
| 6. | Mineral Oil | 3% |
| 7. | Aerosil | 5.0% |
| 8. | Butylated Hydroxy toluene | 0.5% |
| 9. | Tocopheryl acetate | 2.0% |
| 10. | Heptane | q.s |
| | Total | 100% |

### Example 4

| S. No | Name of the ingredient | Percentage w/w |
|---|---|---|
| 1. | Camphor | 1.2% |
| 2. | Menthol | 5.7% |
| 3. | Methyl salicylate | 6.3% |
| 4. | Polyisobutylene | 66.3% |
| 5. | Alicyclic saturated hydrocarbon resin | 10% |
| 6. | Mineral Oil | 3% |
| 7. | Aerosil | 5.0% |
| 8. | Butylated Hydroxy toluene | 0.5% |
| 9. | Tocopheryl acetate | 2.0% |
| 10. | Octane | q.s |

| | |
|---|---|
| Total | 100% |

### Example 5

| S. No | Name of the ingredient | Percentage w/w |
|---|---|---|
| 1. | Camphor | 1.2% |
| 2. | Menthol | 5.7% |
| 3. | Methyl salicylate | 6.3% |
| 4. | Polyisobutylene | 66.3% |
| 5. | Alicyclic saturated hydrocarbon resin | 10% |
| 6. | Mineral Oil | 3% |
| 7. | Aerosil | 5.0% |
| 8. | Butylated Hydroxy toluene | 0.5% |
| 9. | Tocopheryl acetate | 2.0% |
| 10. | Pentane | q.s |
| 11. | Hexane | q.s |

| | |
|---|---|
| Total | 100% |

### Example 6

| S. No | Name of the ingredient | Percentage w/w |
|---|---|---|
| 1. | Camphor | 1.2% |
| 2. | Menthol | 5.7% |
| 3. | Methyl salicylate | 6.3% |
| 4. | Polyisobutylene | 66.3% |
| 5. | Alicyclic saturated hydrocarbon resin | 10% |
| 6. | Mineral Oil | 3% |
| 7. | Aerosil | 5.0% |
| 8. | Butylated Hydroxy toluene | 0.5% |
| 9. | Tocopheryl acetate | 2.0% |
| 10. | Pentane | q.s |
| 11. | Heptane | q.s |

| | |
|---|---|
| Total | 100% |

### Example 7

| S. No | Name of the ingredient | Percentage w/w |
|---|---|---|
| 1. | Camphor | 1.2% |
| 2. | Menthol | 5.7% |
| 3. | Methyl salicylate | 6.3% |
| 4. | Polyisobutylene | 66.3% |
| 5. | Alicyclic saturated hydrocarbon resin | 10% |
| 6. | Mineral Oil | 3% |
| 7. | Aerosil | 5.0% |
| 8. | Butylated Hydroxy toluene | 0.5% |
| 9. | Tocopheryl acetate | 2.0% |
| 10. | Pentane | q.s |
| 11. | Octane | q.s |

| | |
|---|---|
| Total | 100% |

### Example 8

| S. No | Name of the ingredient | Percentage w/w |
|---|---|---|
| 1. | Camphor | 1.2% |
| 2. | Menthol | 5.7% |
| 3. | Methyl salicylate | 6.3% |
| 4. | Polyisobutylene | 66.3% |
| 5. | Alicyclic saturated hydrocarbon resin | 10% |
| 6. | Mineral Oil | 3% |
| 7. | Aerosil | 5.0% |
| 8. | Butylated Hydroxy toluene | 0.5% |
| 9. | Tocopheryl acetate | 2.0% |
| 10. | Hexane | q.s |
| 11. | Heptane | q.s |

| | |
|---|---|
| Total | 100% |

### Example 9

| S. No | Name of the ingredient | Percentage w/w |
|---|---|---|
| 1. | Camphor | 1.2% |
| 2. | Menthol | 5.7% |
| 3. | Methyl salicylate | 6.3% |
| 4. | Polyisobutylene | 66.3% |
| 5. | Alicyclic saturated hydrocarbon resin | 10% |
| 6. | Mineral Oil | 3% |
| 7. | Aerosil | 5.0% |
| 8. | Butylated Hydroxy toluene | 0.5% |
| 9. | Tocopheryl acetate | 2.0% |
| 10. | Hexane | q.s |
| 11. | Octane | q.s |

| | |
|---|---|
| Total | 100% |

### Example 10

| S. No | Name of the ingredient | Percentage w/w |
|---|---|---|
| 1. | Camphor | 1.2% |
| 2. | Menthol | 5.7% |
| 3. | Methyl salicylate | 6.3% |
| 4. | Polyisobutylene | 66.3% |
| 5. | Alicyclic saturated hydrocarbon resin | 10% |
| 6. | Mineral Oil | 3% |
| 7. | Aerosil | 5.0% |
| 8. | Butylated Hydroxy toluene | 0.5% |
| 9. | Tocopheryl acetate | 2.0% |
| 10. | Heptane | q.s |
| 11. | Octane | q.s |

| | |
|---|---|
| Total | 100% |

### Manufacturing Process

An Adhesive base was prepared by first dissolving the combination of hydrophobic high molecular weight & low molecular Polyisobutylene adhesive in organic solvent shown in examples 1-10. Then the mixture of all drugs, Mineral oil, Butylated Hydroxy toluene, Tocopheryl acetate and hydrocarbon resin was added thereto and the mixture was stirred to obtain a uniform blend of adhesive base. Next, this adhesive base was uniformly coat on to the silicone coated release liner and was allowed to dry at low temperature for evaporation of organic solvents. Then this dried adhesive matrix was laminated by 100% polyester Nonwoven or woven fabric. Then the resulting product cut into a desired size to produce an adhesive patch.

### In-Vitro Release Test for Methyl Salicylate:

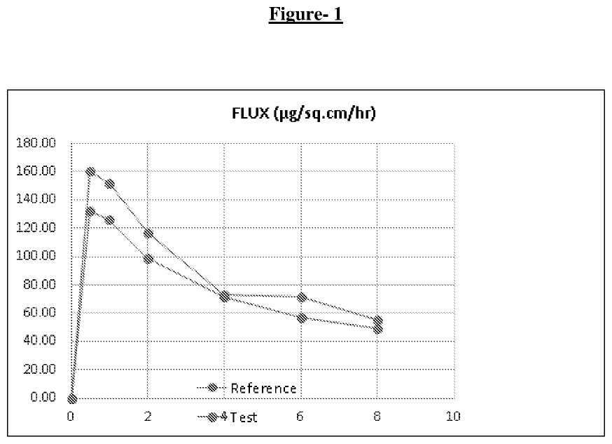

In vitro release test performed through synthetic membrane by using Franz diffusion cell. The 1.539 cm² patch was attached on the synthetic membrane and then placed between the donor and receptor compartments of the cells, with the synthetic membrane side in direct contact with the receptor medium. Approx. 7 ml of the Phosphate buffer (pH 7.4) was placed in the receptor compartment. Its temperature was maintained at 32±0.5° C. using a water bath. This whole assembly was kept on a magnetic stirrer and solution in the receiver compartment was continuously stirred during the whole experiment using magnetic bead. The samples were withdrawn at different time intervals and an equal amount of phosphate buffer (pH 7.4) was replaced each time. Absorbance of the samples were read spectrophotometrically. The amount of methyl salicylate permeated per square centimetre at each time interval was calculated and plotted against time with the receptor medium shown in FIG. 1. The performance verification test (PVT) i.e. 90% confidence interval for Test product has passed with value of 8^{th} value 110.62% & 29^{th} value of 118.57 (Limit: 75 to 133.33) compared with Reference product.

### Reference for PVT Guideline: Scale-Up and Post-approval Changes: Chemistry, Manufacturing, and Controls; In Vitro Release Testing and In Vivo Bioequivalence Documentation.

### U.S. Department of Health and Human Services Food and Drug Administration Centre for Drug Evaluation and Research (CDER), May 1997.

The Residual methyl salicylate content in adhesive matrix after in vitro release study was found to be 52.0%, which is less than reference product i.e. 57.0% for the flux (μg/sq·cm/hr) of methyl salicylate of reference product Vs test product as shown in FIG. 1.
The matric adhesive patch prepared as per the example no. 1 of the present invention is evaluated for the stability at different conditions and the data is given below tables;

TABLE 1

| Product Name: Methyl salicylate, Camphor & Menthol | | | Stability condition: | |
|---|---|---|---|---|
| Topical patch 6.3%, 1.2% & 5.7% | | | 40° C./75% RH | |
| Test | Specifications | Initial | 1 Month | 3 Month |
| Description | Pale orange colored, | Complies | Complies | Complies |
| | opaque, rectangular | | | |
| | shape film. | | | |
| Uniformity of | The Acceptance value | Camphor: | Camphor: | Camphor: |
| Dosage Units (by | is L1 ≤ 15 | 6.36 | 3.55 | 7.72 |
| Content | | Menthol: | Menthol: | Menthol: |
| Uniformity) | | 3.86 | 4.35 | 8.03 |
| | | Methyl | Methyl | Methyl |
| | | salicylate: | salicylate: | salicylate: |
| | | 6.32 | 12.32 | 12.63 |
| Dissolution | NLT 25% of Methyl | 10 Min: 8% | 10 Min: 9% | 10 Min: 9% |
| | salicylate to be | 20 Min: 14% | 20 Min: 14% | 20 Min: 14% |
| | released in 180 min. | 30 Min: 19% | 30 Min: 19% | 30 Min: 18% |
| | | 1 hr: 30% | 1 hr: 30% | 1 hr: 28% |
| | | 2 hr: 43% | 2 hr: 44% | 2 hr: 40% |
| | | 2.5 hr: 47% | 2.5 hr: 47% | 2.5 hr: 44% |
| | | 3 hr: 50% | 3 hr: 50% | 3 hr: 47% |
| Assay | 7.02 mg ± (5.61 to 9.12 | 107.15% | 105.21% | 100.85% |
| Camphor | mg) i.e. 80% to 130% | 112.81% | 105.46% | 104.38% |
| Menthol | 33.34 mg ± (26.67 to | 101.06% | 95.31% | 93.11% |
| Methyl salicylate | 43.34 mg) i.e. 80% to | | | |
| | 130% | | | |
| | 36.85 mg ± (29.48 to | | | |
| | 47.90 mg) i.e. 80% to | | | |
| | 130% | | | |
| Tocopherol | 1.7 mg ± (10.53 to | 104.4% | 99.8% | 95.9% |
| acetate | 12.87 mg) i.e. 90% to 110% | | | |
| content | | | | |
| Residual solvent | NMT 5000 ppm/patch | *ND | **NA | **NA |
| Tack Test | NLT 3.0N | 4.72N | 5.34N | 4.98N |
| Peel Test | NLT 8.0N | 11.42N | 10.61N | 15.35N |
| Release Test | NLT 0.15N | 0.38N | 0.38N | 0.34N |

TABLE 2

| Product Name: Methyl salicylate, Camphor & Menthol | | | Stability condition: | |
|---|---|---|---|---|
| Topical patch 6.3%, 1.2% & 5.7% | | | 30° C./75% RH | |
| Test | Specifications | Initial | 1 Month | 3 Month |
| Description | Pale orange colored, | Complies | Complies | Complies |
| | opaque, rectangular | | | |
| | shape film. | | | |
| Uniformity of | The Acceptance value | Camphor: | Camphor: | Camphor: |
| Dosage Units (by | is L1 ≤ 15 | 6.36 | 13.66 | 10.44 |
| Content | | Menthol: | Menthol: | Menthol: |
| Uniformity) | | 3.86 | 10.93 | 6.86 |
| | | Methyl | Methyl | Methyl |
| | | salicylate: | salicylate: | salicylate: |
| | | 6.32 | 12.23 | 10.17 |
| Dissolution | NLT 25% of Methyl | 10 Min: 8% | **NA | 10 Min: 8% |
| | salicylate to be | 20 Min: 14% | | 20 Min: 14% |
| | released in 180 min. | 30 Min: 19% | | 30 Min: 19% |
| | | 1 hr: 30% | | 1 hr: 31% |
| | | 2 hr: 43% | | 2 hr: 44% |
| | | 2.5 hr: 47% | | 2.5 hr: 49% |
| | | 3 hr: 50% | | 3 hr: 51% |
| Assay | 7.02 mg ± (5.61 to 9.12 | 107.15% | 103.36% | 100.11% |
| Camphor | mg) i.e. 80% to 130% | 112.81% | 110.28% | 101.79% |
| Menthol | 33.34 mg ± (26.67 to | 101.06% | 100.15% | 98.80% |
| Methyl salicylate | 43.34 mg) i.e. 80% to | | | |
| | 130% | | | |
| | 36.85 mg ± (29.48 to | | | |
| | 47.90 mg) i.e. 80% to | | | |
| | 130% | | | |
| Tocopherol | 1.7 mg ± (10.53 to | 104.4% | 97.3% | 92.9% |
| acetate | 12.87 mg) i.e. 90% to | | | |
| content | 110% | | | |
| Residual solvent | NMT 5000 ppm/patch | *ND | **NA | **NA |
| Tack Test | NLT 3.0N | 4.72N | **NA | 5.07N |
| Peel Test | NLT 8.0N | 11.42N | **NA | 14.10N |
| Release Test | NLT 0.15N | 0.38N | **NA | 0.29N |

TABLE 3

| Product Name: Methyl salicylate, Camphor & Menthol | | | Stability condition: | |
|---|---|---|---|---|
| Topical patch 6.3%, 1.2% & 5.7% | | | 25° C./60% RH | |
| Test | Specifications | Initial | 1 Month | 3 Month |
| Description | Pale orange colored, | Complies | Complies | Complies |
| | opaque, rectangular | | | |
| | shape film. | | | |
| Uniformity of | The Acceptance value | Camphor: | Camphor: | Camphor: |
| Dosage Units (by | is L1 ≤ 15 | 6.36 | 11.79 | 6.55 |
| Content | | Menthol: | Menthol: | Menthol: |
| Uniformity) | | 3.86 | 12.89 | 8.60 |
| | | Methyl | Methyl | Methyl |
| | | salicylate: | salicylate: | salicylate: |
| | | 6.32 | 13.34 | 7.67 |
| Dissolution | NLT 25% of Methyl | 10 Min: 8% | NA | 10 Min: 6% |
| | salicylate to be | 20 Min: 14% | | 20 Min: 11% |
| | released in 180 min. | 30 Min: 19% | | 30 Min: 16% |
| | | 1 hr: 30% | | 1 hr: 26% |
| | | 2 hr: 43% | | 2 hr: 38% |
| | | 2.5 hr: 47% | | 2.5 hr: 42% |
| | | 3 hr: 50% | | 3 hr: 46% |
| Assay | 7.02 mg ± (5.61 to 9.12 | 107.15% | 101.98% | 98.37% |
| Camphor | mg) i.e. 80% to 130% | 112.81% | 107.16% | 103.75% |
| Menthol | 33.34 mg ± (26.67 to | 101.06% | 98.10% | 97.91% |
| Methyl salicylate | 43.34 mg) i.e. 80% to | | | |
| | 130% | | | |
| | 36.85 mg ± (29.48 to | | | |
| | 47.90 mg) i.e. 80% to | | | |
| | 130% | | | |
| Tocopherol | 1.7 mg ± (10.53 to | 104.4% | 99.2% | 93.8% |
| acetate | 12.87 mg) i.e. 90% to | | | |
| content | 110% | | | |
| Residual solvent | NMT 5000 ppm/patch | *ND | **NA | **NA |
| Tack Test | NLT 3.0N | 4.72N | **NA | 5.15N |
| Peel Test | NLT 8.0N | 11.42N | **NA | 10.72N |
| Release Test | NLT 0.15N | 0.38 | **NA | 0.24N |

*NA: Not applicable,
**ND: Not Detected

1. A matrix adhesive patch composition comprising Camphor, Menthol, Methyl salicylate and one or more other excipients.

2. The matrix patch composition as claimed in claim 1, wherein the one or more other excipients is selected from adhesive material, tackifying resin, adsorbent material and antioxidants.

3. The matrix patch composition as claimed in claim 1, wherein the composition is produced by a solvent evaporation technique to reduce the loss of drugs during manufacturing process.

4. The matrix patch composition as claimed in claim 1, wherein the composition comprises a low boiling solvent to minimize the drying temperature and time, this leads to reduce the drug loss from the adhesive matrix during drying process.

5. The matrix patch composition as claimed in claim 1, wherein the composition is produced by a process for preparing a matrix type patch composition, wherein the process comprising steps of:
a. dissolving the combination of hydrophobic high molecular weight & low molecular adhesive material in one or more organic solvents,
b. adding one or more active ingredients, tackifying resins, antioxidants by stirring the mixture to obtain a uniform blend of adhesive base,
c. coating the adhesive base uniformly on to the silicone coated release liner and drying at low temperature for evaporation of organic solvents,
d. laminating the dried adhesive matrix by 100% polyester Nonwoven or woven fabric, and
e. cutting the resulting product into a desired size to producing an adhesive patch.

6. The matrix patch composition as claimed in claim 2, wherein adhesive material referred above preferably include hydrophilic polymers include, light anhydrous silicic acid, cellulose derivatives [carboxymethyl cellulose (CMC), carboxymethyl cellulose sodium (CMCNa), methyl cellulose (MC), hydroxypropyl methylcellulose (HPMC), hydroxypropyl cellulose (HPC), hydroxyethyl cellulose (HEC)], starch derivatives (pullulan), polyvinyl alcohol (PVA), polyvinylpyrrolidone (PVP), vinyl acetate (VA), carboxyvinyl polymer (CVP), ethylvinyl acetate (EVA), Eudragit, gelatin, polyacrylic acid, sodium polyacrylate, polyisobutylene, combination of hydrophobic Low & high molecular weight polyisobutylene, polyisobutylene-maleic anhydride copolymer, alginic acid, sodium alginate, carrageenan, Arabian gum, tragacanth gum, mineral oil, karaya gum and polyvinyl methacrylate.

7. The matrix patch composition as claimed in claim 2, wherein tackifying resin referred above preferably includes an alicyclic saturated hydrocarbon resin, a poly-terpene resin, petroleum resin, rosin, rosin ester and fat soluble phenol resin or combinations thereof.

8. The matrix patch composition as claimed in claim 2, wherein adsorbent material referred above includes colloidal silica, Aerosil, talc Al₂O₃, Ti0₂, MgO, synthetic and amorphous silicas, Syloid, and synthetic and amorphous silicates and combinations thereof.

9. The matrix patch composition as claimed in claim 2, wherein antioxidant referred above includes butylhydroxytoluene (BHT), butylhydroxyanisole (BHA), tertiary-butylhydroquinone (TBHQ), Tocopheryl acetate, alpha tocopherol, ascorbic-acid, ascorbyl palmitate, propyl gallate, fumaric acid, malic acid, sodium ascorbate, sodium metabisulfite and combinations thereof.

10. The matrix patch composition as claimed in claim 1, wherein the composition further comprise one or more excipients selected from plasticizers, solvents, stabilizer, surfactant, reducing agent, base, filler, coloring matter, softener, excipient, antiseptics, preservative, solubilizing agent, solubilizer, solvent, super plasticizer, antistatic agent, extender and moisturizing agent.
